(19) United States
(12) Patent Application Publication
(10) Pub. No.: US 2010/0249465 A1
(43) Pub. Date: Sep. 30, 2010

(54) PROCESS FOR PRODUCTION OF HIGH-PURITY VINYL ETHER

(75) Inventors: Ryuichi TENJIMBAYASHI, Chiba (JP); Ichiro Kimura, Chiba (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: MARUZEN PETROCHEMICAL CO., LTD., Chuo-ku (JP)

(21) Appl. No.: 12/729,639

(22) Filed: Mar. 23, 2010

(30) Foreign Application Priority Data
Mar. 26, 2009 (JP) 2009-076033

(51) Int. Cl. C07C 41/42

(52) U.S. Cl. 568/689; 568/579

(57) ABSTRACT

A process for producing a high-purity vinyl ether, which comprises:
a step of subjecting an alcohol represented by the general formula (1)

R—O—H (1)

to a vinyl ether formation reaction in the presence of a catalyst to synthesize a vinyl ether represented by the general formula (2),

R—O—CH═CH₂ (2)
a step of removing the catalyst from the reaction mixture obtained in the above step to obtain a crude vinyl ether containing the vinyl ether and the unreacted raw material alcohol,
a step of reacting the unreacted raw material alcohol in the crude vinyl ether, with the vinyl ether in the presence of an acid catalyst, to convert the alcohol into an acetal represented by the general formula (3),

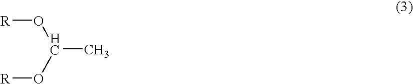

and
a step of subjecting a crude vinyl ether containing the acetal (III) to distillation to obtain a high-purity vinyl ether.

### BACKGROUND OF THE INVENTION

1. Field of the Invention

The present invention relates to a process for production of a vinyl ether. More particularly, the present invention relates to a process for production of a high-purity vinyl ether from a raw material alcohol, in which the unreacted raw material alcohol in the crude vinyl ether can be removed efficiently.

2. Description of the Prior Art

Vinyl ether can be produced by an addition reaction of alcohol to acetylene, an ether exchange reaction between vinyl ether and alcohol, using as a catalyst, a transition metal complex or the like, or a vinyl exchange reaction between vinyl carboxylate and alcohol.

In any of these processes for production of vinyl ether, an alcohol is used as a raw material. However, separation and recovery of the produced vinyl ether by distillation is difficult when the unreacted raw material alcohol in the crude vinyl ether forms an azeotropic mixture with the vinyl ether.

In order to solve such a problem, there are reported a method in which azeotropy is destroyed by addition of alkali metal salt (Patent Literature 1) and a method in which, utilizing a phenomenon that azeoptropic composition differs depending upon the pressure of the system, two distillation columns are used and distillation is carried out under different pressures to remove the unreacted raw material alcohol (Patent Literatures 2 and 3, etc.).

However, in the method in which azeotropy is destroyed by addition of alkali metal salt, there is a problem that the reaction of alkali metal salt with alcohol produces an alkali metal alcoholate and the alcoholate separates out as a solid in the distillation column bottom residue due to a decrease of the reaction mixture, making the method unusable as a industrial process.

Also, in the method in which two distillation columns are used and distillation is carried out under different pressures, the pressure inside the second distillation column is made higher than the pressure inside the first distillation column, whereby there is taken out, from the top of the second distillation column, an azeotropic mixture having an alcohol concentration slightly higher than that of the azeotropic mixture distilling from the first distillation column and there is recovered a vinyl ether from the bottom of the second distillation column; however, since main component of the azeotropic mixture taken out from the top of the second distillation column is also the vinyl ether, there is a problem that the yield of vinyl ether becomes low.

### [Prior Art Literatures]

Patent Literature 1: U.K. Patent No. 787915
Patent Literature 2: JP-A-1998-109952
Patent Literature 3: National Publication of International Patent Application No. 2006-527225

### SUMMARY OF THE INVENTION

The present invention was made in order to solve the above-mentioned problems of the prior art. The present invention has a task to provide a process for production of a high-purity vinyl ether, which can recover a high-purity vinyl ether efficiently from a crude vinyl ether containing the unreacted raw material alcohol even when the vinyl ether and the alcohol form an azeotropic mixture and which includes a purification step of easy handling.

The present inventors made a study in order to achieve the above task. As a result, it was found that the unreacted raw material alcohol in the crude vinyl ether can be converted into an acetal by addition of a very small amount of an acid catalyst with the polymerization of the vinyl ether being suppressed and the above conversion of the unreacted raw material alcohol into acetal makes it possible to obtain, by distillation, a high-purity vinyl ether easily. The finding has led to the completion of the present invention.

The present invention achieves the above task by the inventions described in the following [1] to [8].
[1] A process for producing a high-purity vinyl ether, which comprises:
   a first step; a vinyl ether synthesis step of subjecting an alcohol (I) represented by the general formula (1)

R—O—H (1)

(wherein R is an aliphatic hydrocarbon group or an alicyclic hydrocarbon group), to a vinyl ether formation reaction in the presence of a catalyst to synthesize a vinyl ether (II) represented by the general formula (2)

R—O—CH═CH₂ (2)

(wherein R is an aliphatic hydrocarbon group or an alicyclic hydrocarbon group)
a second step; a catalyst removal step of removing the catalyst from the reaction mixture obtained in the first step to obtain a crude vinyl ether containing the vinyl ether (II) and the unreacted raw material alcohol (I)
a third step; an acetal formation step of reacting the unreacted raw material alcohol (I) in the crude vinyl ether, with the vinyl ether (II) in the presence of an acid catalyst, to convert the alcohol (I) and vinyl ether (II) into an acetal (III) represented by the general formula (3)

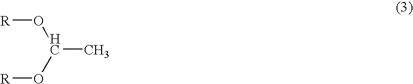

(wherein R is an aliphatic hydrocarbon group or an alicyclic hydrocarbon group), and
a fourth step; a distillation and purification step of subjecting a crude vinyl ether containing the acetal (III) to distillation to obtain a high-purity vinyl ether.
[2] A process for producing a high-purity vinyl ether according to [1], wherein the vinyl ether formation reaction is a reaction selected from the following reactions:
   (A) an ether exchange reaction between a vinyl ether and an alcohol,
   (B) a vinyl exchange reaction between a vinyl carboxylate and an alcohol, and
   (C) an addition reaction of an alcohol to acetylene.
[3] A process for producing a high-purity vinyl ether according to [1] or [2], wherein the second step is a step of subjecting the reaction mixture of the vinyl ether formation reaction to distillation to remove the catalyst, and obtaining a crude vinyl ether containing the alcohol (I) and the vinyl ether (II).
[4] A process for producing a high-purity vinyl ether according to [1] or [2], wherein the second step includes, as a part of the step, an operation of subjecting the mixture obtained by the catalyst removal from the reaction mixture of the vinyl ether formation reaction to distillation, and obtaining a crude vinyl ether containing the alcohol (I) and the vinyl ether (II).
[5] A process for producing a high-purity vinyl ether according to any of [1] to [4], wherein, in the third step, the acid catalyst used in the acetal formation reaction is an inorganic acid, organic acid or solid acid catalyst.
[6] A process for producing a high-purity vinyl ether according to any of [1] to [5], wherein, in the third step, the acetal formation reaction is carried out at 0 to 80° C.
[7] A process for producing a high-purity vinyl ether according to any of [1] to [6], wherein the third step includes, as a part of the step, an operation of neutralizing and/or removing the acid catalyst after the acetal formation reaction.
[8] A process for producing a high-purity vinyl ether according to any of [1] to [7], which comprises recovering the distillation column bottom residue rich in the acetal (III), converting the acetal (III) into the alcohol (I) and the vinyl ether (II), and then recycling the alcohol (I) and the vinyl ether (II) to the vinyl ether synthesis step as raw materials for vinyl ether synthesis.

According to the production process of the present invention, a high-purity vinyl ether can be obtained easily by distillation, by, in the crude vinyl ether containing a vinyl ether and an unreacted raw material alcohol, converting the unreacted raw material alcohol into an acetal even when the vinyl ether and the alcohol form an azeotropic mixture. Further, the acetal contained in the distillation column bottom residue is recovered, and converted into a vinyl ether and a raw material alcohol, and can be recycled as raw materials for vinyl ether synthesis; thus, a vinyl ether can be produced efficiently.

### BRIEF DESCRIPTION OF THE DRAWING

FIG. 1 is a schematic view showing an embodiment of the production process of the present invention.

### EXPLANATION OF NUMERAL SYMBOLS

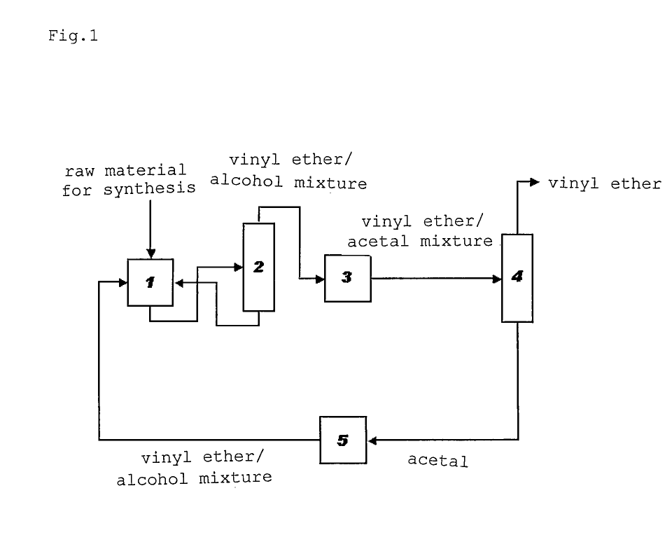

In FIG. 1, 1 indicates a vinyl ether synthesis step; 2 indicates a catalyst removal step; 3 indicates an acetal formation step; 4 indicates a distillation and purification step; and 5 indicates an acetal decomposition step.

### DETAILED DESCRIPTION OF THE INVENTION

The outline of an embodiment of the present invention is described based on FIG. 1. A reaction mixture containing an unreacted raw material alcohol (I), a vinyl ether (II) and a catalyst, obtained in the first step (vinyl ether synthesis step) is fed to the second step (catalyst removal step), the third step (acetal formation step) and the fourth step (distillation and purification step) in this order, and a high-purity vinyl ether is obtain in the fourth step.

### [1] First Step: Vinyl Ether Synthesis Step

In the first step, an alcohol (I) represented by the general formula (1)

R—O—H (1)

is subjected to a vinyl ether formation reaction in the presence of a catalyst to synthesize a vinyl ether (II) represented by the general formula (2)

R—O—CH═CH₂ (2)

to obtain a reaction mixture containing the vinyl ether (II), the unreacted raw material alcohol (I) and the catalyst.

In the general formulas (1) and (2), R is an aliphatic hydrocarbon group or an alicyclic hydrocarbon group, preferably an aliphatic hydrocarbon group of 2 to 10 carbon atoms or an alicyclic hydrocarbon group of 3 to 10 carbon atoms.

As specific examples of the aliphatic hydrocarbon group, there can be mentioned straight chain or branched chain alkyl groups such as ethyl group, n-propyl group, isopropyl group, butyl group, isobutyl group, sec-butyl group, tert-butyl group, pentyl group, isopentyl group, neopentyl group, n-hexyl group, n-heptyl group, n-octyl group, n-nonyl group, n-decyl group, 2-ethylhexyl group and the like; alkyl groups having cycloalkyl substituent, such as cyclohexylmethyl group, tricyclodecanylmethyl group, 1-adamantylmethyl group and the like; and straight chain or branched chain alkenyl groups such as allyl group, isopropenyl group, butenyl group, pentenyl group, hexenyl group, heptenyl group, octenyl group and the like.

As specific examples of the alicyclic hydrocarbon group, there can be mentioned monocyclic cycloalkyl groups such as cyclopropyl group, cyclobutyl group, cyclopentyl group, cyclohexyl group, cyclooctyl group and the like; monocyclic cycloalkenyl groups such as cyclopentenyl group, cyclohexenyl group and the like; and polycyclic hydrocarbon groups such as perhydronaphthyl group, adamantyl group, tricyclodecanyl group, norbornyl group and the like.

As specific examples of the alcohol (I) used as a raw material, there can be mentioned straight chain or branched chain saturated aliphatic alcohols such as ethanol, n-propanol, isopropanol, n-butanol, isobutanol, sec-butanol, tert-butanol, n-pentanol, isopentanol, neopentanol, n-hexanol, n-heptanol, n-octanol, n-nonanol, n-decanol, 2-ethylhexanol and the like; straight chain or branched chain unsaturated aliphatic alcohols such as allyl alcohol, isopropenyl alcohol, butenyl alcohol, pentenyl alcohol, hexenyl alcohol, heptenyl alcohol, octenyl alcohol and the like; and alicyclic alcohols such as cyclopentanol, cyclohexanol, tricyclodecanol, 1-adamantanol, 2-adamntnaol, cyclohexanemethanol and the like.

As specific examples of the vinyl ether (II) obtained, there can be mentioned straight chain or branched chain alkyl vinyl ethers such as ethyl vinyl ether, n-propyl vinyl ether, isopropyl vinyl ether, n-butyl vinyl ether, isobutyl vinyl ether, sec-butyl vinyl ether, tert-butyl vinyl ether, n-pentyl vinyl ether, isopentyl vinyl ether, neopentyl vinyl ether, n-hexyl vinyl ether, n-heptyl vinyl ether, n-octyl vinyl ether, n-nonyl vinyl ether, n-decyl vinyl ether, 2 ethylhexyl vinyl ether and the like; straight chain or branched chain alkenyl vinyl ethers such as allyl vinyl ether, isopropenyl vinyl ether, butenyl vinyl ether, pentenyl vinyl ether, hexenyl vinyl ether, heptenyl vinyl ether, octenyl vinyl ether and the like; and alicyclic vinyl ethers such as cyclopentyl vinyl ether, cyclohexyl vinyl ether, tricyclodecane vinyl ether, 1-adamantane vinyl ether, 2-adamantane vinyl ether, cyclohexylmethyl vinyl ether and the like.

Particularly, the present invention can be used effectively in a system where the raw material alcohol (I) represented by the general formula (1) and the vinyl ether (II), represented by the general formula (2) form an azeotropic mixture.

The vinyl ether (II) can be prepared from the alcohol (I) represented by the general formula (1), as raw material, by a known method. As specific methods, there can be mentioned:
(A) an ether exchange method between a vinyl ether and an alcohol,
(B) a vinyl exchange method between a vinyl carboxylate and an alcohol, and
(C) an addition reaction of an alcohol to acetylene.

In the ether exchange reaction (A), the alcohol (I) is reacted with a vinyl ether (IV) different from the intended vinyl ether (II), represented by the general formula (4)

R¹—O—CH═CH₂ (4)

(wherein R¹ is an aliphatic hydrocarbon group or an alicyclic hydrocarbon group) in the presence of a transition metal complex catalyst, to obtain a vinyl ether (II).

As the raw material vinyl ether (IV), there is used a vinyl ether which is easily procured as compared with the intended vinyl ether (II). Specifically, there are preferably used methyl vinyl ether, ethyl vinyl ether, n-propyl vinyl ether, n-butyl vinyl ether, etc.

As the transition metal complex catalyst, there can be used a known catalyst for the vinyl ether exchange reaction, such as palladium complex and cobalt complex. As specific examples of the palladium complex, there can be mentioned 1,10-phenanthroline complexes of palladium such as palladium acetate-1,10-phenanthroline complex, palladium chloride-1,10-phenanthroline complex and the like. As specific examples of the cobalt complex, there can be mentioned cobalt carbonyl complexes such as Co(CH₃COCHCOCH₃)₂, CO(CH₃COCHCOCH₃)₃, Co(CH₃COCHCOCH₃)₂.2H₂O, CO₂(CO)₈ and the like.

In the ether exchange reaction (A), an organic solvent may be used. As the organic solvent, there can be mentioned, for example, saturated hydrocarbon solvents such as pentane, hexane, heptane, cyclopentane, cyclohexane and the like; ethers such as dioxane, diethyl ether, diisopropyl ether, methyl tert-butyl ether, tetrahydrofuran, sulfolane, diethylene glycol dimethyl ether, diethylene glycol diethyl ether, triethylene glycol dimethyl ether, triethylene glycol diethyl ether and the like; and aromatic hydrocarbon solvents such as benzene, toluene and the like.

The reaction temperature is ordinarily −20 to 150° C. and, from the standpoints of reaction rate and side reaction suppression, preferably 0 to 100° C., more preferably 20 to 50° C. The reaction time differs depending upon the reaction conditions but is ordinarily about 10 minutes to 48 hours.

In the vinyl exchange reaction (B), the alcohol (I) is reacted with a vinyl carboxylate (V) represented by the following general formula (5)

R²COO—CH═CH₂ (5)

(wherein R² is an aliphatic hydrocarbon group, an alicyclic hydrocarbon group or an aromatic hydrocarbon group) in the presence of a transition metal complex catalyst and a basic compound, to obtain a vinyl ether (II).

As specific examples of the vinyl carboxylate (V) used as a raw material, there can be mentioned vinyl acetate, vinyl propionate, vinyl formate and vinyl benzoate.

As the transition metal complex catalyst, there can be used a known catalyst for vinyl exchange reaction, such as iridium complex. As specific examples of the iridium complex, there can be mentioned di-μ-chlorotetrakis(cyclooctene) diiridium (I), di-μ-chlorotetrakis(ethylene) diiridium (I), di-μ-chlorobis(1,5-cyclooctadiene) diiridium (I), bis(1,5-cyclooctadiene) iridium tetrafluoroborate, and (1,5-cyclooctadiene) (acetonitrile) iridium tetrafluoroborate.

As the basic compound, there can be mentioned, for example, hydroxides, carbonates and hydrogencarbonates of alkali metals such as sodium, potassium and the like.

Since the vinyl exchange reaction is an equilibrium reaction, it is preferred to carry out the reaction in the presence of a basic compound, in order to capture the carboxylic acid formed by vinyl removal. It is further preferred to carry out the reaction while removing the water generated by the reaction between the carboxylic acid and the basic compound, out of the system.

In the vinyl exchange reaction (B), an organic solvent may be used. As the organic solvent, there can be mentioned, for example, saturated hydrocarbon solvents such as pentane, hexane, heptane, cyclopentane, cyclohexane and the like; ethers such as dioxane, diethyl ether, diisopropyl ether, methyl tert-butyl ether, tetrahydrofuran, sulfolane, diethylene glycol dimethyl ether, diethylene glycol diethyl ether, triethylene glycol dimethyl ether, triethylene glycol diethyl ether and the like; and aromatic hydrocarbon solvents such as benzene, toluene and the like.

The reaction temperature is ordinarily 50 to 170° C. and, from the standpoints of reaction rate and side reaction suppression, preferably 70 to 150° C., more preferably 90 to 130° C. The reaction time differs depending upon the reaction conditions but is ordinarily about 10 minutes to 48 hours.

In the reaction (C) (addition reaction of alcohol to acetylene), the alcohol (I) is reacted with acetylene in the presence of an alkali metal alcoholate catalyst, to obtain a vinyl ether (II).

The alkali metal alcoholate catalyst is a compound which is synthesized from the raw material alcohol (I) and one compound selected from sodium hydroxide, potassium hydroxide, rubidium hydroxide and cesium hydroxide or a mixture thereof. The catalyst is preferred to be soluble in the alcohol (I) for handling.

In the reaction (C), an organic solvent may be used. Preferred as the organic solvent is, for example, an aprotic polar solvent which is miscible with the raw material alcohol and dissolves the alkali metal alcoholate catalyst. Specifically, there are used, for example, amide type solvents such as dimethylacetamide, 2-pyrrolidone, N-methyl-2-pyrrolidne, 1,3-dimethyl-2-imidazolidinone and the like; sulfur-containing compound type solvents such as sulfolane, dimethyl sulfoxide and the like; and glycol dialkyl ether type solvents such as diethylene glycol dimethyl ether, diethylene glycol diethyl ether, triethylene glycol dimethyl ether, triethylene glycol diethyl ether and the like.

The reaction temperature is ordinarily 80 to 200° C. and, from the standpoints of reaction rate and side reaction suppression, preferably 100 to 180° C. With respect to the reaction pressure, a higher pressure gives a larger reaction rate but a pressure of 0.3 MPa or lower is preferred in order to prevent the decomposition and explosion of acetylene. The reaction time differs depending upon the reaction conditions but is ordinarily about 10 minutes to 48 hours.

In the present invention, of the reactions (A) to (C), the reaction (C) (addition reaction of alcohol to acetylene) is preferably employed because it gives a high yield, uses inexpensive raw materials and requires no catalyst.

### [2] Second Step: Catalyst Removal Step

In the second step, the catalyst used in the reaction is removed from the reaction mixture after vinyl ether formation reaction, to obtain a crude vinyl ether containing the vinyl ether (II) and the unreacted raw material alcohol (I).

The removal of catalyst can be carried out by a known method, for example, solid and liquid separation (in the case of solid catalyst or supported catalyst) such as solvent extraction, distillation, filtration or the like. Of these methods, distillation is preferred because it is easy to separate the catalyst and can allow for decrease in alcohol concentration.

Even when the catalyst is removed by a method other than distillation, it is preferred to conduct distillation to lower the alcohol content in crude vinyl ether.

The distillation column used in the distillation may be any of packed column, plate column, bubble cap column, etc. The plate number of the distillation column is, for example, 1 to 100, preferably 5 to 50 in terms of theoretical plate number. The pressure in distillation is ordinarily 0.7 to 13.3 kPa, preferably 1.3 to 6.7 kPa.

### [3] Third Step: Acetal Formation Step

In the third step, the unreacted raw material alcohol (I) in the crude vinyl ether obtained in the second step is reacted with the vinyl ether (II) in the presence of an acid catalyst, to convert the alcohol into an acetal (III) represented by the general formula (3)

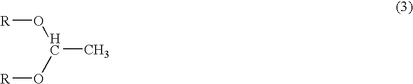

[wherein R is the same as in the general formula (1) and the general formula (2)].

As the acid catalyst used in the acetal formation reaction, there can be mentioned, for example, inorganic acids such as sulfuric acid, nitric acid, hydrochloric acid, phosphoric acid and the like; organic acids such as carboxylic acid, organic sulfonic acid and the like; and solid acid catalysts such as acidic zeolite, heteropoly-acid, strongly acidic ion exchange resin and the like.

Of these acid catalysts, preferred are phosphoric acid, organic sulfonic acid, sulfonic acid group-containing strongly acidic ion exchange resin, etc. from the standpoint of side reaction suppression, in particular, suppression of vinyl ether polymerization.

As the organic sulfonic acid, there can be mentioned aromatic sulfonic acids such as p-toluenesulfonic acid, o-toluenesulfonic acid, benzenesulfonic acid, p-xylene-2-sulfonic acid, dodecylbenzenesulfonic acid, 1-naphthalenesulfonic acid, 2-naphthalenesulfonic acid, dinonylnaphthalenesulfonic acid, dinonylnaphthalenedisulfonic acid and the like; aliphatic sulfonic acids such as methanesulfonic acid, ethanesulfonic acid, trifluoromethanesulfonic acid and the like; aromatic sulfonic acid salts such as p-toluenesulfonic acid pyridinium salt, p-toluenesulfonic acid quinolinium salt and the like; and so forth.

As the strongly acidic ion exchange resin, there can be mentioned sulfonic acid type strongly acidic ion exchange resins such as Amberlyst 15 DRY (trade name, a product of Organo) and the like; a mixture of sulfonic acid type strongly acidic ion exchange resin [e.g. Amberlyst MSPS 2-1•DRY (trade name, a product of Organo)] and amine type weakly basic ion exchange resin; and so forth.

The use amount of the acid catalyst differs depending upon the kind of the acid used and therefore cannot be determined in a specified range. However, the use amount is ordinarily 0.1 to 3,000 ppm relative to the crude vinyl ether, preferably 0.5 to 2,000 ppm, more preferably 1 to 1,000 ppm when an inorganic acid is used as the acid catalyst; when an organic acid is used as the acid catalyst, the use amount is ordinarily 1 to 3,000 ppm relative to the crude vinyl ether, preferably 5 to 2,000 ppm, more preferably 10 to 1,000 ppm; when a solid acid catalyst is used as the acid catalyst, the use amount is ordinarily 0.01 to 5.0 mass % relative to the crude vinyl ether, preferably 0.5 to 3.0 mass %, more preferably 0.1 to 1.0 mass % With a too large amount of acid catalyst, side reactions such as polymerization of vinyl ether and the like may take place; with a too small amount, a sufficient reaction rate may not be obtained.

The inorganic acid or the organic acid may be used neat or dissolved in an appropriate solvent. The solid acid catalyst may be added directly to the crude vinyl ether; or it may be filled in a column or the like, followed by passing the crude vinyl ether therethrough for acetal formation.

The temperature for acetal formation is preferably 0 to 80° C., more preferably 10 to 60° C. from the standpoints of reaction rate and side reaction suppression. The reaction time differs depending upon the reaction conditions employed but is ordinarily about 10 minutes to 48 hours.

Incidentally, it is preferred that, after the acetal formation reaction, the acid catalyst is neutralized and/or removed because the remaining of the acid catalyst may cause formation of heavy material in the next step (the distillation and purification step).

When an inorganic acid or an organic acid is used as the acid catalyst, the neutralization is desirably carried out by addition of a basic compound. As the basic compound, there can be mentioned alkali metal compounds, for example, hydroxides, carbonates and hydrogencarbonates of alkali metals (e.g. sodium or potassium); basic ion exchange resins; etc. The basic compound is used preferably in an amount excessive relative to the acid catalyst.

The basic compound, when it is an alkali metal compound, may be used neat or dissolved in an appropriate solvent and, when it is a basic ion exchange resin, may be added directly or may be filled in a column or the like, followed by passing the crude vinyl ether therethrough for neutralization. When a solid or a precipitate is present in the solution after the neutralization, the solution may be, as necessary, separated into solid and liquid by filtration, centrifugation or the like.

Meanwhile, when a solid acid catalyst is used as the acid catalyst, the neutralization is ordinarily unnecessary and solid and liquid separation is carried out by filtration, centrifugation or the like. When the solid acid catalyst is used by being filled in a column or the like, no separation is necessary.

### [4] Fourth Step: Distillation and Purification Step

In the fourth step, the crude vinyl ether containing the acetal (III) is subjected to distillation to obtain a high-purity vinyl ether.

The acetal (III) and the vinyl ether (II) are largely different in boiling point and form no azeotropic mixture; therefore, they can be easily separated by distillation and a high-purity vinyl ether of 99 mass % or higher in purity can be obtained efficiently.

As to the distillation apparatus or method used, there is no particular restriction, and any of simple distillation and plate distillation may be employed. When plate distillation is employed, the theoretical plate number is ordinarily 1 to 20, preferably 5 to 10. The pressure during distillation is ordinarily 0.1 to 13.3 kPa, preferably 0.1 to 6.7 kPa.

An intended high-purity vinyl ether is obtained from the top of distillation column and a bottom residue rich in acetal (III) is recovered from the bottom of the distillation column.

### [5] Fifth Step: Acetal Decomposition Step

The bottom residue rich in acetal (III), obtained in the fourth step is recovered as necessary and the acetal (III) is converted into an alcohol (I) and a vinyl ether (II); then, they can be recycled to the vinyl ether synthesis step as raw materials for vinyl ether synthesis.

A known method can be used for the conversion of the acetal (III) into an alcohol (I) and a vinyl ether (II), and there is no particular restriction. Specifically, there can be mentioned, for example,
(a) a method by thermal decomposition in a gaseous phase in the presence of a silica/alumina type catalyst loaded with an alkali or alkaline earth metal [for example, Khim. Prom. 48 (9) 657-660, 1972; JP-A-1973-78109; JP-A-1987-87247],
(b) a method by decomposition in a gaseous phase using magnesium oxide as a catalyst (JP-A-1996-268945),
(c) a method by decomposition in the presence of a noble metal-containing catalyst (for example, Ann., 60181-84, 1956; German Laid-Open Patent No. 1957680; JP-A-1973-76803), and
(d) a method by decomposition using an acid catalyst [for example, J. Org. Chem., 38, 2910, 1973, Helv. Chim. Acta, 1158 (1967); Bull. Chem. Soc. Jpn. 3089 (1976); JP-A-1996-277237]

### EXAMPLES

Hereinafter, the present process is described by way of Examples. However, these Examples are for illustration and do not restrict the present invention.

### Example 1

### Production Example I of High-Purity 2-ethylhexyl vinyl ether

### (Vinyl Ether Synthesis Step and Catalyst Removal Step)

4,800 g of 2-ethylhexanol and 500 g of potassium hydroxide were taken by metering, into a continuous reaction and distillation apparatus equipped with a reaction vessel of 10 liters (internal volume) and a distillation column of 10 theoretical plates. Dehydration was performed under reduced pressure while heating at 120° C., to prepare a potassium alcoholate catalyst. Then, acetylene (20 kPa, 103 g/h) and 2-ethylhexanol (557 g/h) were fed continuously; a reaction was carried out at 137° C.; and there was obtained, from the top of the distillation column, a crude 2-ethylhexyl vinyl ether (686 g/h, composition: 2-ethylhexyl vinyl ether 90 mass % and 2-ethylhexanol 10 mass %).
(Acetal formation step and distillation and purification step)

346.8 g of the crude 2-ethylhexyl vinyl ether obtained in the above steps was taken by metering, into a 500-ml, three-necked flask equipped with a stirrer bar. Thereto was added 0.29 g (711 ppm relative to the crude vinyl ether) of a 85% aqueous phosphoric acid solution, followed by stirring at room temperature for 60 minutes for a reaction. In the mixture after the reaction, the content of 2-ethylhexanol was 0.1 mass % or less and there were 80 mass % of 2-ethylhexyl vinyl ether and 20 mass % of acetaldehyde di(2-ethylhexyl) acetal. To the reaction mixture was added 4.94 g of a methanol solution containing 8.8 mass % of potassium hydroxide, for neutralization. Then, using a simple distillation apparatus, distillation was carried out (inside pressure: 4.6 kPa, oil bath temperature set: 95 to 120° C., coolant temperature set: 5° C.) to obtain 245.1 g (recovery yield: 70.7%) of high-purity 2-ethylhexyl vinyl ether of 99 mass % or higher in purity.

### Example 2

### Production Example II of High-Purity 2-ethylhexyl vinyl ether)

1,507 g of a crude 2-ethylhexyl vinyl ether obtained in the same manner as in Example 1 was taken by metering, into a 2-liter, three-necked flask equipped with a stirrer bar. Thereto was added 47.7 mg (31.5 ppm relative to the crude 2-ethylhexyl vinyl ether) of p-toluenesulfonic acid monohydrate, followed by stirring at room temperature for 10 minutes for a reaction. In the mixture after the reaction, the content of 2-ethylhexanol was 0.1 mass % or less and there were 80 mass % of 2-ethylhexyl vinyl ether and 20 mass % of acetaldehyde di(2-ethylhexyl) acetal. To the reaction mixture was added 47.7 mg of a saturated aqueous sodium hydroxide solution, for neutralization. Then, using a 10-plate, Oldershaw-type distillation apparatus, distillation was carried out (inside pressure: 1.3 kPa, oil bath temperature set: 70 to 136° C., coolant temperature set: 20° C.) to obtain 1,044 g (recovery yield: 69.3%) of high-purity 2-ethylhexyl vinyl ether of 99 mass % or higher in purity.

### Example 3

### Production Example III of High-Purity 2-ethylhexyl vinyl ether

### (Vinyl Ether Synthesis Step and Catalyst Removal Step)

5,015 g of 2-ethylhexanol and 500 g of potassium hydroxide were taken by metering, into a continuous reaction and distillation apparatus equipped with a reaction vessel of 10 liters (internal volume) and a distillation column of 10 theoretical plates. Dehydration was performed under reduced pressure while heating at 120° C., to prepare a potassium alcoholate catalyst. Then, acetylene (20 kPa, 65 g/h) and 2-ethylhexanol (361 g/h) were fed continuously; a reaction was carried out at 140° C.; and there was obtained, from the top of the distillation column, a crude 2-ethylhexyl vinyl ether (401 g/h, composition: 2-ethylhexyl vinyl ether 94 mass % and 2-ethylhexanol 6 mass %).

### (Acetal Formation Step and Distillation and Purification Step)

87.2 g of the crude 2-ethylhexl vinyl ether obtained in the above steps and 0.15 g (0.17 mass % relative to the crude 2-ethylhexyl vinyl ether) of Amberlyst 15 DRY (trade name) were taken by metering, into a 200-ml, Erlenmeyer flask equipped with a stirrer bar, followed by stirring at room temperature for 20 minutes for a reaction. In the mixture after the reaction, the content of 2-ethylhexanol was 0.1 mass % or less and there were 88 mass % of 2-ethylhexyl vinyl ether and 12 mass % of acetaldehyde di(2-ethylhexyl) acetal. The Amberlyst 15 DRY was removed by filtration, after which distillation was carried out using an evaporator (inside pressure: 0.2 kPa, oil bath temperature set: 50° C., coolant temperature set: 0° C.) to obtain 65.8 g (recovery yield: 75.5%) of high-purity 2-ethylhexyl vinyl ether of 99 mass % or higher in purity.

### Example 4

### Production Example I of High-Purity cyclohexyl vinyl ether

### (Vinyl Ether Synthesis Step and Catalyst Removal Step)

2,500 g of cyclohexanol, 2,500 g of triethylene glycol dimethyl ether as a solvent and 250 g of potassium hydroxide were taken by metering, into a continuous reaction and distillation apparatus equipped with a reaction vessel of 10 liters (internal volume) and a distillation column of 10 theoretical plates. Dehydration was performed by nitrogen bubbling while heating at 120° C., to prepare a potassium alcoholate catalyst. Then, acetylene (20 kPa, 138 g/h) and cyclohexanol (529 g/h) were fed continuously; a reaction was carried out at 135° C.; and there was obtained, from the top of the distillation column, a crude cyclohexyl vinyl ether (636 g/h, composition: cyclohexyl vinyl ether 95 mass %, cyclohexanol 4 mass %, and other impurity 1 mass %).

### (Acetal Formation Step and Distillation and Purification Step)

268.7 g of the crude cyclohexyl vinyl ether obtained in the above steps and 0.71 g (0.26 mass % relative to the crude cyclohexyl vinyl ether) of Amberlyst 15 DRY (trade name) were taken by metering, into a 500-ml, Erlenmeyer flask equipped with a stirrer bar, followed by stirring at room temperature for 10 minutes for a reaction. In the mixture after the reaction, the content of cyclohexanol was 0.1 mass % or less and there were 91 mass % of cyclohexyl vinyl ether and 8 mass % of acetaldehyde dicyclohexyl acetal (the remainder was other impurity). The Amberlyst 15 DRY was removed by filtration, after which distillation was carried out using an evaporator (inside pressure: 0.1 kPa, oil bath temperature set: 25° C., coolant temperature set: 0° C.) to obtain 201.8 g (recovery yield: 75.1%) of high-purity 2-cyclohexyl vinyl ether of 99 mass % or higher in purity.

What is claimed is:

1. A process for producing a high-purity vinyl ether, which comprises:
a first step; a vinyl ether synthesis step of subjecting an alcohol (I) represented by the general formula (1)
R—O—H (1)
(wherein R is an aliphatic hydrocarbon group or an alicyclic hydrocarbon group), to a vinyl ether formation reaction in the presence of a catalyst to synthesize a vinyl ether (II) represented by the general formula (2)
R—O—CH═CH₂ (2)
(wherein R is an aliphatic hydrocarbon group or an alicyclic hydrocarbon group)
a second step; a catalyst removal step of removing the catalyst from the reaction mixture obtained in the first step to obtain a crude vinyl ether containing the vinyl ether (II) and the unreacted raw material alcohol (I)
a third step; an acetal formation step of reacting the unreacted raw material alcohol (I) in the crude vinyl ether, with the vinyl ether (II) in the presence of an acid catalyst, to convert the alcohol (I) and vinyl ether (II) into an acetal (III) represented by the general formula (3) 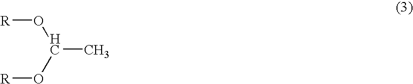
(wherein R is an aliphatic hydrocarbon group or an alicyclic hydrocarbon group), and
a fourth step; a distillation and purification step of subjecting a crude vinyl ether containing the acetal (III) to distillation to obtain a high-purity vinyl ether.

2. A process for producing a high-purity vinyl ether according to claim 1, wherein the vinyl ether formation reaction is a reaction selected from the following reactions:
(A) an ether exchange reaction between a vinyl ether and an alcohol,
(B) a vinyl exchange reaction between a vinyl carboxylate and an alcohol, and
(C) an addition reaction of an alcohol to acetylene.

3. A process for producing a high-purity vinyl ether according to claim 1 or 2, wherein the second step is a step of subjecting the reaction mixture of the vinyl ether formation reaction to distillation to remove the catalyst, and obtaining a crude vinyl ether containing the alcohol (I) and the vinyl ether (II).

4. A process for producing a high-purity vinyl ether according to claim 1 or 2, wherein the second step includes, as a part of the step, an operation of subjecting the mixture obtained by the catalyst removal from the reaction mixture of the vinyl ether formation reaction to distillation, and obtaining a crude vinyl ether containing the alcohol (I) and the vinyl ether (II).

5. A process for producing a high-purity vinyl ether according to any of claims 1 to 4, wherein, in the third step, the acid catalyst used in the acetal formation reaction is an inorganic acid, organic acid or solid acid catalyst.

6. A process for producing a high-purity vinyl ether according to any of claims 1 to 5, wherein, in the third step, the acetal formation reaction is carried out at 0 to 80° C.

7. A process for producing a high-purity vinyl ether according to any of claims 1 to 6, wherein the third step includes, as a part of the step, an operation of neutralizing and/or removing the acid catalyst after the acetal formation reaction.

8. A process for producing a high-purity vinyl ether according to any of claims 1 to 7, which comprises recovering the distillation column bottom residue rich in the acetal (III), converting the acetal (III) into the alcohol (I) and the vinyl ether (II), and then recycling the alcohol (I) and the vinyl ether (II) to the vinyl ether synthesis step as raw materials for vinyl ether synthesis.
